(19) United States
(12) Patent Application Publication
(10) Pub. No.: US 2023/0338169 A1
(43) Pub. Date: Oct. 26, 2023

(54) DEVICES AND METHODS TO HARVEST ELECTRICAL ENERGY FROM ELECTROMOTIVE FORCE

(71) Applicant: LOMA LINDA UNIVERSITY HEALTH, Loma Linda, CA (US)

(72) Inventors: Chelsie K. Rodgers, Loma Linda, CA (US); Abraham Castillo, Loma Linda, CA (US); Michael Davidson, Loma Linda, CA (US)

(73) Assignee: LOMA LINDA UNIVERSITY HEALTH, Loma Linda, CA (US)

(21) Appl. No.: 18/041,250

(22) PCT Filed: Aug. 10, 2021
(86) PCT No.: PCT/US21/45338

§ 371 Date: Feb. 10, 2023

Related U.S. Application Data
(60) Provisional application No. 63/063,477, filed on Aug. 10, 2020.

(51) Int. Cl. A61F 2/70; A61F 2/66

(52) U.S. Cl. CPC A61F 2/70; A61F 2/66; A61F 2002/708

(57) ABSTRACT

A device for harvesting electrical energy from movement of a prosthetic limb. The device including a generator and a controller. The generator adapted to attach to a prosthetic limb. The generator comprising a magnetic linear motor that is configured to generate electrical energy in response to movement of a portion of the prosthetic limb. The controller is electrically connected to the generator and configured to provide electrical energy to a power supply of the prosthetic limb.

### CROSS-REFERENCE TO RELATED APPLICATION

This application claims priority to, and benefit of, U.S. Provisional Patent Application Ser. No. 63/063,477, filed Aug. 10, 2020. The entire contents of this application are hereby incorporated by reference.

### FIELD OF DISCLOSURE

This disclosure relates generally to electrical scavenging systems, and in particular, to devices for harvesting electrical energy from certain electromotive forces and using that electrical energy to power a medical device or to charge a battery within a medical device.

### BACKGROUND

In recent years, the need for artificial limbs (i.e., prostheses) has grown significantly due, for example, to increases in armed conflicts around the world and medical interventions (e.g., the treatment of peripheral arterial disease). At the same time, technical innovation has made prostheses much more comfortable, efficient, and capable. Generally, a prosthesis (e.g., a leg) is designed with moveable and adjustable joints, motors, and pylons which replicate the functions of a human limb.

Prosthetic devices can include one or more powered devices that require electrical energy to operate. The powered devices of prosthetic devices can be simple or very complex in design. These devices operate off a built-in power source and can be programmed to the user. The power sources are charged and attached to the wearer before the wearer goes about their day. The physical requirements of these prostheses include generating torque to replicate what the human body is normally responsible for. Due to this demand, the motor(s) used within the prosthesis can require a lot of electrical energy. As such, the motor(s) of prosthesis can drain the power source (e.g., battery) rather quickly. The power source may provide a prosthesis with about four to six hours of use. When the power source gets low, the wearer is left searching for an outlet and waiting for the power source to charge, or worse.

### SUMMARY

This disclosure relates generally to a device for harvesting electrical energy to charge and/or operate a medical device, such as a prosthetic limb. Such a device may increase a usage time or completely eliminate the need to externally charge a power source of the prosthetic limb.

In one aspect, the disclosure is related to a device to harvest electrical energy from an electromotive force generated by movement of a prosthetic limb. The device includes a generator and a controller. The generator is positioned to generate electrical energy when connected to a prosthetic limb and is responsive to movement of the prosthetic limb. The controller is electrically connected to the generator and a power supply of the prosthesis. The generator is connected to at least a portion of the prosthetic limb and includes a magnetic linear motor connected to at least a portion of the prosthetic limb. The motor is configured so that movement of the prosthetic limb causes the generator to generate electrical energy in the form of alternating current. The controller is configured to convert the alternating current generated by the generator into direct current. The direct current may be used to charge the power supply; however, in other embodiments, the direct current may also be stored or used to directly power the prosthetic limb.

In another aspect, the disclosure relates to a device to harvest electrical energy from an electromotive force generated by movement of a prosthetic limb. The device includes a generator positioned to generate electrical energy when connected to a prosthetic limb and a controller electrically connected to the generator and a power supply of the prosthesis. The generator is connected to at least a portion of the prosthetic limb and includes a magnetic linear motor actuated via movement of the prosthetic limb during a swing phase of a human gait, thereby causing the generator to generate electrical energy in the form of alternating current. The controller includes a current converter to convert the alternating current generated by the generator into direct current for charging the power supply. In some embodiments, the current converter includes a bridge rectifier.

In various embodiments of the foregoing aspects, the magnetic linear motor includes a hollow tubular body, at least one coil of wire wrapped about a portion of the hollow tubular body, and a first magnet slidably disposed within the hollow tubular body. The first magnet slides within the tubular body and through the at least one coil of wire during a swing phase of a human gait, thereby inducing the alternating current within the coil of wire. The generation of the electrical energy may be substantially constant during movement of the prosthetic limb. The magnetic linear motor may also include a second magnet disposed in a first end cap connected to a first end of the hollow tubular body and a third magnet disposed within a second end cap connected to a second end of the hollow tubular body. The second and third magnets may be oriented such that their polarities are opposite a corresponding polarity of the first magnet slidably disposed therebetween. In some embodiments, each of the first, second, and third magnets include neodymium magnets, and the first magnet may be larger than the second and third magnets.

In further embodiments, the at least one coil of wire wrapped about a portion of the hollow tubular body includes four separate coils of wire in electrical communication and spaced approximately equidistant along a length of the hollow tubular body. In certain embodiments, the wire may be a 22 gauge enameled copper wire. The controller may include a bridge rectifier configured to convert the alternating current into the direct current, an electrical storage medium electrically connected to the bridge rectifier and positioned to receive the direct current for storage thereof, or both. In certain embodiments, the storage medium may output the generated electrical energy stored therein as needed by the power supply of the prosthetic limb. In some embodiments, the device operates like a trickle charger to maintain the charge of the prosthetic limb's power supply and/or may store the electrical energy for later use, for example, in the event of a malfunction or complete discharge of the limb's power supply. The electrical storage medium may include at least one of a battery or a capacitor. Additionally, the controller may include a microprocessor configured to control one or more operations of at least one of the device or the prosthetic limb. The controller may also include a display configured to display an amount of energy available from the storage medium.

Furthermore, the generator may include a first generator and a second generator, each positioned to generate electrical energy when connected to the prosthetic limb and responsive to movement of the prosthetic limb. The second generator may be used to generate additional electrical energy in the form of alternating current and may be connected to a second portion of the prosthetic limb and the controller. In some cases, the second generator includes a second magnetic linear motor, while in others, the second generator includes a piezo-electric motor. In certain embodiments, the prosthetic limb is a prosthetic leg, where the first generator is disposed within or adjacent a foot when positioned on a user and the second generator is disposed within or adjacent a heel of the foot when positioned on a user. The size and shape of the generator may vary to suit a particular application, and in some embodiments, a length of the generator substantially corresponds to a length of the prosthetic limb. Maximizing the length of a magnetic linear motor maximizes a distance that the first magnet may travel within the motor and an amount electrical energy generated.

In yet another aspect, the disclosure relates to a method of harvesting electrical energy from an electromotive force generated by movement of a prosthetic limb. The method includes the steps of connecting a generator to the prosthetic limb, where the generator is responsive to a movement of the prosthetic limb; moving the prosthetic limb through at least a portion of a swing phase of a human gait; and generating electrical energy in the form of alternating current through the movement of at least a portion of the generator relative to the prosthetic limb.

In various embodiments, the generator includes a housing fixedly connected to the prosthetic limb and at least one component movably disposed within the housing. Movement of the prosthetic limb causes the at least one component to move within the housing, thereby generating the alternating current. In certain embodiments, the generator includes at least one coil of wire disposed about the housing and the at least one component includes at least one magnet slidably disposed within the housing. The at least one magnet slides within the housing and through the at least one coil of wire during at least a portion of a swing phase of a human gait, thereby inducing the alternating current within the at least one coil of wire. Additionally, the method may include the steps of connecting a controller to at least a portion of the prosthetic limb; electrically connecting the controller to the generator and a power supply of the prosthesis; directing the alternating current to the controller; using the controller to convert the alternating current to a direct current (e.g., via a current converter, such as a bridge rectifier); and transmitting the direct current to the power supply of the prosthetic limb. In some cases, at least a portion of the direct current may be stored for later use. Additionally, the method may include connecting a second generator to the prosthetic limb.

In another embodiment of the present disclosure, a device to harvest electrical energy from movement of a prosthetic limb includes a generator and a controller. The generator is adapted to attach to a prosthetic limb and includes a first motor configured to generate electrical energy in response to movement of a portion of the prosthetic limb. The controller is electrically connected to the generator and is configured to provide electrical energy to a power supply of the prosthetic limb. The controller may be configured to charge the power supply of the prosthetic limb.

In embodiments, the first motor is configured to be disposed within the portion of the prosthetic limb. The first motor may be in the form of a magnetic linear motor and include a hollow tubular body, at least one coil of wire wrapped about the body, and a first magnet slidably disposed within the body. The first magnet may be configured to slide within the body and through the at least one coil of wire in response to movement of the portion of the prosthetic limb such that electrical energy is generated in the at least one coil of wire. The first magnet may be configured to slide within the body during a swing phase of a human gait.

In some embodiments, the first motor includes a first end cap, a second end cap, a second magnet, and a third magnet. The first end cap may close a first end of the body and the second end cap may close a second end of the body opposite the first end. The second magnet may be disposed in the first end cap and the third magnet may be disposed in the second end cap. The second and third magnets may be oriented such that polarities thereof oppose polarities of the first magnet slidably disposed therebetween. The first magnet, second magnet, or third magnet may comprise a neodymium magnet. The first magnet may be larger than the second magnet and/or the third magnet.

In embodiments, the first end cap closing the first end of the body and a second end cap closing a second end of the body. The first end cap includes a first bumper that is secured to the first end cap and disposed within the body. The second end cap includes a second bumper that is secured to the second end cap and is disposed within the body. The first bumper and the second bumper are configured to engage the first magnet.

In certain embodiments, the at least one coil of wire forms four separate coils of wire wrapped about the body. The Four separate coils may be in electrical communication with one another and may be spaced equidistant along the body.

In particular embodiments, the first motor is configured to generate electrical energy in the form of alternating current. The controller may be configured to convert the alternating current from the generator to direct current and to provide the direct current to the power supply of the prosthetic. The controller may comprise a bridge rectifier that is configured to convert the alternating current to direct current. The controller may include an electrical storage medium that is electrically connected to the bridge rectifier. The electrical storage medium may be configured to receive and store electrical energy from the bridge rectifier. The electrical storage medium may be configured to output the stored electrical energy as direct current to the power supply of the prosthetic limb. The electrical storage medium may include a battery or a capacitor.

In embodiments, the controller includes a microprocessor that is configured to control one or more operations of the device of the prosthetic limb. The generator may include a second motor that is configured to generate electrical energy in response to movement of the prosthetic limb. The second motor may be electrically connected to the controller. The second motor may be a magnetic linear motor or a piezo-electric motor.

In another embodiment of the present disclosure, a prosthetic limb includes a first portion, a power supply, and a generator. The generator includes a first motor that is configured to generate electrical energy in response to movement of the first portion. The controller is electrically connected to the generator and is configured to provide electrical energy to the power supply.

In embodiments, the first motor is a magnetic linear motor or a piezo-electric motor.

The first motor may be disposed within the first portion.

In some embodiments, the generator includes a second motor that is configured to generate electrical energy in response to movement of the prosthetic limb. The first motor and the second motor may be disposed in the first portion.

In particular embodiments, the prosthetic limb includes a second portion and the second motor is configured to generate electrical energy in response to movement of the second portion of the prosthetic limb. The prosthetic limb may include a foot with the second portion being the heel of the foot. The prosthetic limb may include a leg segment with the first portion being a second of the leg segment.

In another embodiment of the present disclosure, a device to harvest electrical energy from movement of a prosthetic limb includes a generator and a controller. The generator is positioned to generate electrical energy when connected to a prosthetic limb. The generator includes a magnetic linear motor that is configured to be actuated via movement of the prosthetic limb during a swing phase of a human gait to generate electrical energy. The controller is electrically connected to the generator and is configured to provide electrical energy to a power supply of the prosthetic limb.

In embodiments, the magnetic linear motor is configured to generate electrical energy in the form of alternating current and the controller is configured to convert the alternating current to direct current.

In another embodiment of the present disclosure, a method of harvesting electrical energy from movement of a prosthetic limb includes connecting a generator to the prosthetic limb, moving the prosthetic limb through at least a portion of a swing phase of a human gait, and generating electrical energy with the generator during the swing phase. The generator generates electrical energy via movement of a component of the generator relative to the prosthetic limb.

In embodiments, connecting the generator to the prosthetic limb includes fixedly connecting a housing of the generator to the prosthetic limb and movably disposing the component within the housing. Generating electrical energy may include the movement of the component generating electrical energy in the form of alternating current. Generating electrical energy may include the component comprising a magnet slidably disposed within the housing of the generator. The generator may include at least one coil of wire disposed about the housing such that movement of the magnet within the housing induces electrical energy in the form of alternating current in the at least one coil of wire.

In some embodiments, the method includes connecting the controller to a power supply of the prosthetic limb, configuring electrical energy in the form of alternating current provided to the controller from the generator to direct current within the controller and transmitting the direct current to the power supply of the prosthetic limb.

Still other aspects, embodiments, and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Accordingly, these and other objects, along with advantages and features of the present disclosure herein disclosed, will become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the disclosure and are not intended as a definition of the limits of the disclosure. For purposes of clarity, not every component may be labeled in every drawing. In the following description, various embodiments of the present disclosure are described with reference to the following drawings, in which:

FIG. 1A is a perspective view of a device for harvesting electrical energy from an electromotive force in accordance with one or more embodiments of the disclosure;

FIG. 1B is a top view of the device of FIG. 1A in accordance with one or more embodiments of the disclosure;

FIG. 2 is a perspective view of a generator for use in a device for harvesting electrical energy from an electromotive force in accordance with one or more embodiments of the disclosure;

FIG. 3 is an exploded view of the generator of FIG. 2;

FIG. 4 is a wire-frame, perspective view of the generator of FIG. 2;

FIG. 5A is a perspective view of a controller for a device for harvesting electrical energy from an electromotive force in accordance with one or more embodiments of the disclosure;

FIG. 5B is a top view of the controller of FIG. 5A in accordance with one or more embodiments of the disclosure;

FIGS. 6A and 6B are electrical schematics for a device for harvesting electrical energy from an electromotive force in accordance with one or more embodiments of the disclosure;

FIG. 7 is a representation of a prosthetic limb for discussion purposes;

FIG. 8 is a schematic side view of a device for harvesting electrical energy from an electromotive force installed within a prosthetic leg in accordance with one or more embodiments of the disclosure;

FIG. 9 is a perspective view of another embodiment of a magnetic linear motor provided in accordance with an embodiment of the present disclosure with some internal portions shown;

FIG. 10 is a longitudinal cross-sectional view of the motor of FIG. 9; and

FIG. 11 is an exploded view of the motor of FIG. 9.

### DETAILED DESCRIPTION

The present disclosure will now be described more fully hereinafter with reference to example embodiments thereof with reference to the drawings in which like reference numerals designate identical or corresponding elements in each of the several views. These example embodiments are described so that this disclosure will be thorough and complete, and will fully convey the scope of the disclosure to those skilled in the art. Features from one embodiment or aspect can be combined with features from any other embodiment or aspect in any appropriate combination. For example, any individual or collective features of method aspects or embodiments can be applied to apparatus, product, or component aspects or embodiments and vice versa. The disclosure may be embodied in many different forms and should not be construed as limited to the embodiments set forth herein; rather, these embodiments are provided so that this disclosure will satisfy applicable legal requirements. As used in the specification and the appended claims, the singular forms “a,” “an,” “the,” and the like include plural referents unless the context clearly dictates otherwise. In addition, while reference may be made herein to quantitative measures, values, geometric relationships or the like, unless otherwise stated, any one or more if not all of these may be absolute or approximate to account for acceptable variations that may occur, such as those due to manufacturing or engineering tolerances or the like.

Generally, the devices disclosed herein relate to capturing electrical energy generated by an electromotive force to operate a medical device or store electrical energy for later use. Electromotive Force (EMF), which may be measured in volts, is the electrical action produced by a non-electrical source. For example, EMF may be movement of the human body captured by a generator and converted into electrical energy. Some of the generators disclosed herein utilize electromagnetic induction, which involves moving a magnet through the middle of a wire coil to generate an electrical current in the wire. The current generated flows in the direction that the magnet moves such that an alternating current is generated as the magnet moves back and forth through the coil(s). In various embodiments, the generators may be incorporated into or retrofitted to a prosthetic limb such that movement of the limb causes movement of the magnets relative to one or more coils to generate electrical energy in the form of alternating current (AC).

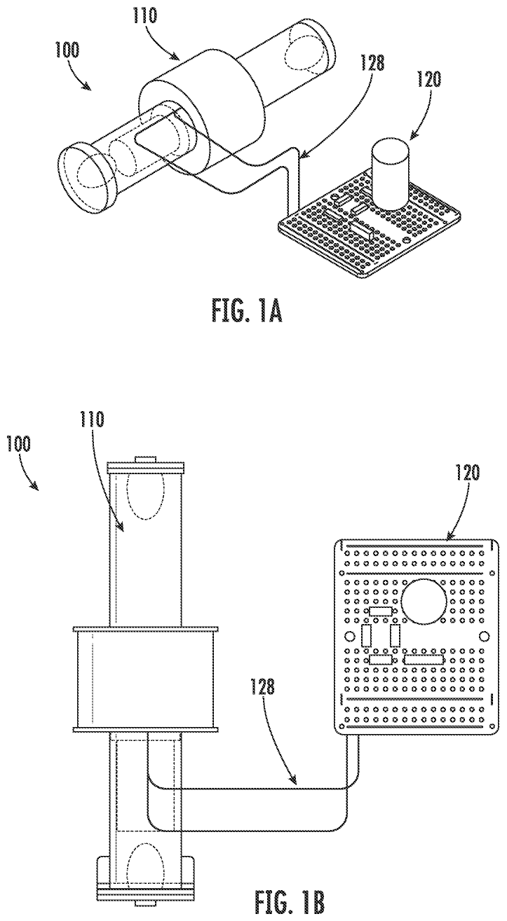

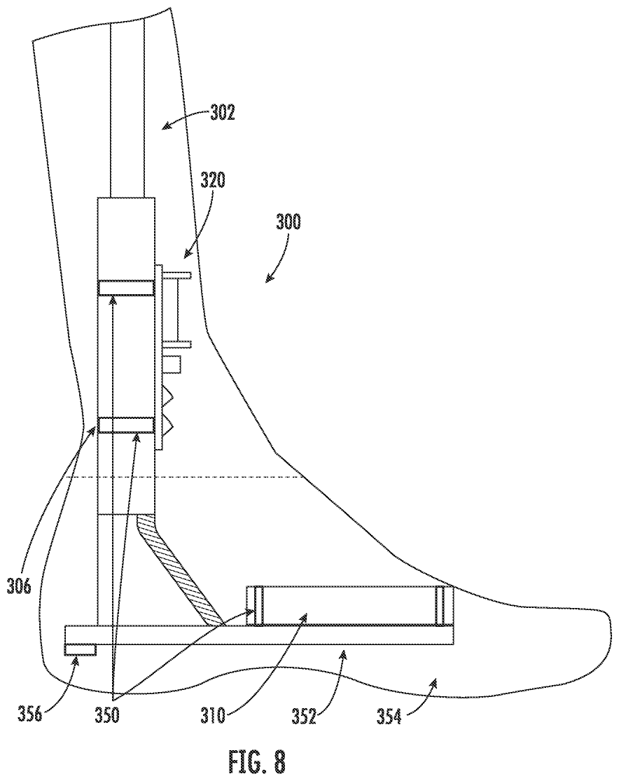

FIGS. 1A and 1B depict one embodiment of a device 100 for harvesting electrical energy from an electromotive force generated by movement of a prosthetic limb. As shown, the device 100 includes a generator 110 and a controller 120 electrically connected thereto. The device 100 is shown in an uninstalled state and the physical relationship between the components may vary to suit a particular installation, as described below with respect to FIG. 8. The generator 110 may be sized and shaped to fit within at least a portion of the prosthetic limb, as may the controller 120, as will be understood by those skilled in the art. The controller 120 may be electrically connected to and may be in electrical communication with the generator 110 via two (2) wires 128 as described below. The wires 128 may be 22 gauge wires. The controller 120 receives electrical energy in the form of alternating current generated by the generator 110 and converts the AC to direct current (DC) that may be directed to one of the powered devices within the prosthesis 202. In some embodiments, the electrical energy may be stored for later use (e.g., as a back-up to the main power source of the prosthesis or as a trickle charger to keep the main prosthesis battery charged).

FIGS. 2-4 depict various views of a generator 110 in accordance with one or more disclosed embodiments. As shown, the generator 110 is a magnetic linear motor; however, other configurations of the generator 110 may fall within the scope of this disclosure. The generator 110 includes a substantially cylindrical housing or body 112 upon which one or more wire coils 118 may be placed thereabout and within which a plurality of magnets 114 may be disposed. In some embodiments, the magnetic linear motor may be replaced by or supplemented with a piezo-electric motor that generates electrical energy based on a change of shape in the motor's material, somewhat like a voice coil, as will be understood by those skilled in the art.

The housing, for example, may be made from a hollow tubular body 112 enclosed on its ends with end caps 114 attached thereto. The end caps 114 may be affixed or bonded to the body 112 or may be removably attached to the body 112 (e.g., threadably attached). The end caps 114 secure the magnets 116 within the body 112 such that the magnets 116 are prevented from exiting the tubular body 112. The body 112 and/or end caps 114 may be made of polyvinyl chloride (PVC) or other polymeric materials. In some embodiments, the body 112 and/or the end caps 114 may be made from other non-metallic materials. The diameter and length of the tubular body 112, and thus the generator 110 overall, will vary to suit a particular application (e.g., type of prosthesis and size of prosthesis). In some embodiments, the body 112 may have a length of 3 inches to 24 inches, alternatively 6 inches to 12 inches. In some embodiments, the tubular body 112 and end caps 114 may have a nominal diameter of 0.25 inches to 2 inches, alternatively 0.5 inches. In the embodiments shown, the tubular body 112 has a circular cross-sectional shape; however, the cross-sectional shape of the tubular body 112 may vary to suit a particular application (e.g., location within the prosthesis, mounting requirements, etc.) and may include, but not be limited to, rectangular, trapezoidal, square, hexagonal, or oval.

The generator 110 includes at least one wire coil 118 wrapped around the tubular body 112. In the embodiment shown, a single wire is wrapped around the body 112 to form four (4) coils 118 that are spaced substantially equidistant apart. The generator 110 may include a range of 1 to 10 coils 118. The coils 118 may be formed of a single wire or multiple wires. In some embodiments, a single coil 118 may include multiple wires that are wrapped around the tubular body 112 together. The specific number of coils 118, the number of wires, and the spacing therebetween may vary to suit a particular application, with the number of coils 118 and length of the wire influencing the voltage generated. In various embodiments, the coils are formed from a 22 or 32 gauge enameled copper magnet wire that shields the copper wire from the elements. The enameled copper magnet wire may allow for better flow of electrons. The enameled copper magnet wire may require less insulation such that a magnetic field may be improved compared to a non-enameled copper magnet wire. The enamel may be removed as necessary with a fine grit file.

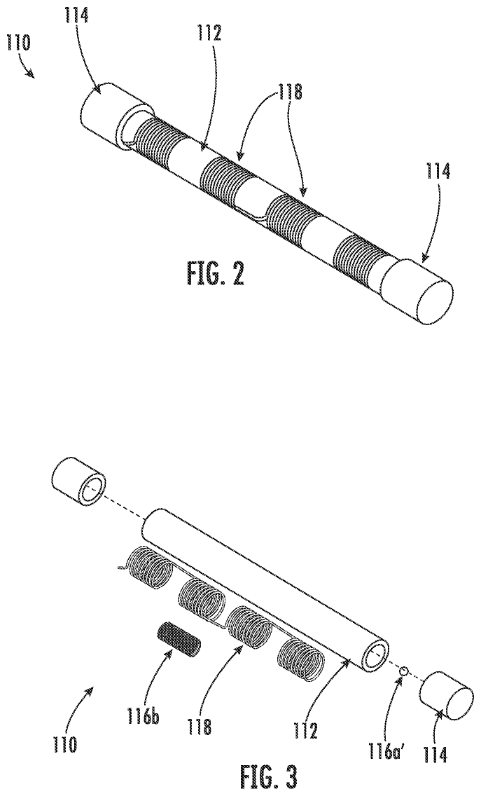

As shown in FIGS. 3 and 4, the generator 110 includes three (3) magnets 116. Two of the magnets 116a are disposed within the end caps 114 and are secured therein by, for example, an adhesive or a mechanical fastener as will be understood by those skilled in the art, while the third magnet 116b is slidably disposed within the tubular body 112. In various embodiments, the magnets 116a in the end caps 114 are oriented so that their surfaces facing away from the end caps 114 are of opposite polarities. Specifically, the first magnet 116a may be attached to an end cap 114 so that its north pole is oriented towards the space defined by the body 112, while the second magnet 116a′ may be attached to its corresponding end cap 114 so that its south pole is oriented towards the space defined by the body 112. The third magnet 116b is oriented within the space defined by the body 112 so that its north pole opposes the north pole of the first magnet 116a and its south pole opposes the south pole of the second magnet 116a′. Because like polarities (i.e., two north poles or two south poles) repel one another, the magnets 116a in the end caps 114 will act as springs. The size, shape, and strength of the magnets 114 will be selected to suit a particular application (e.g., size or configuration of the prosthesis 202), with the third magnet 116b in particular being sized and shaped to correspond to the size and shape of the body 112, so as to freely slide therein. In some embodiments, the magnets 116 are neodymium magnets, with the magnets 116a within the end caps 114 being smaller (e.g., 6 mm×3 mm) than the magnet 116b slidably disposed within the tubular body 112. The larger magnet 116b should be sized (e.g., 15 mm×3 mm) so that it may pass through substantially the entire length of the tubular body 112 to generate the electrical energy.

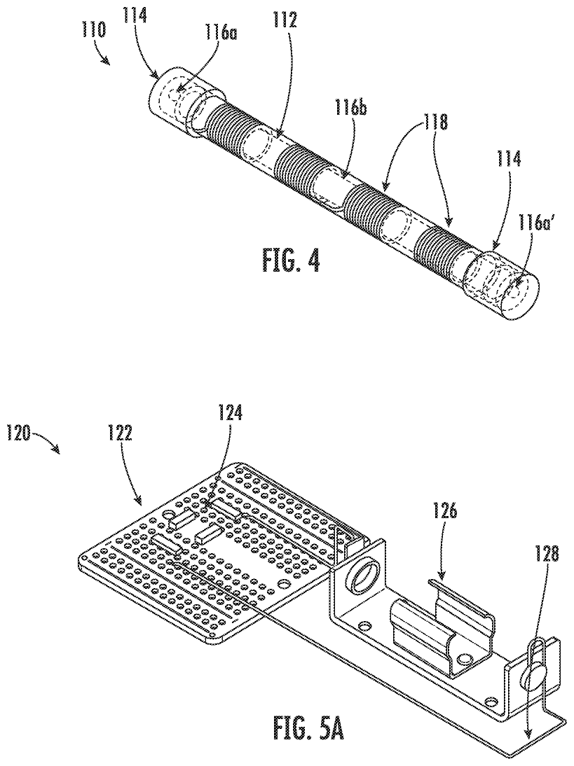

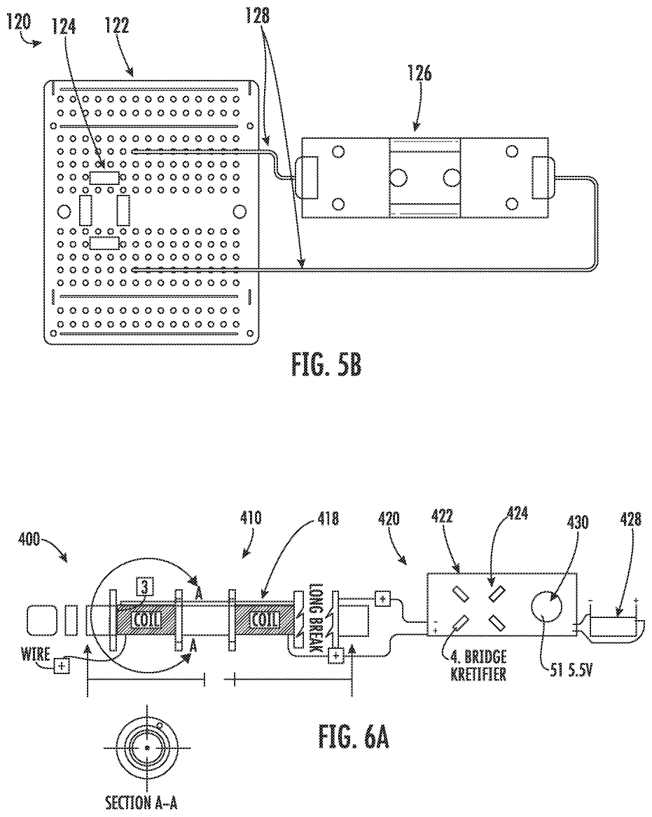

FIGS. 5A and 5B depict an exemplary controller 120 for use in any of the devices described herein. The controller 120 includes a circuit board 122, a bridge rectifier 124, and a storage medium or device 126, all electrically connected via any necessary wiring 128. The circuit board 122 may support other electrical components (e.g., a microprocessor) as may be included in various embodiments of the device 100. The bridge rectifier 124 may be soldered to the circuit board 122 followed by any necessary wiring 128. In some embodiments, the bridge rectifier 124 may be configured as an H-circuit of four diodes for taking in electrical energy in the form of alternating current from the generator 110 and converting the electrical energy to direct current that may be stored or used to directly power some portion of the prosthesis.

The storage device (or medium) 126 of the controller 120 may take on various forms and is generally configured to receive electrical energy in the form of direct current from the rectifier 124. In some embodiments, the storage device may be a battery mount (e.g., configured for receiving one or more AA batteries) to which a battery may be attached. In various embodiments, the storage device 126 is a Ni—Cd 850 mAh (1.2 v) AA rechargeable battery. In embodiments, the storage device 126 is a capacitor soldered to the circuit board 122. The wiring 128 may be standard 22-gauge wire soldered to the enameled copper wire to transfer the electrical energy from the generator 110 to the bridge rectifier 124 and it may be soldered to the hot (power) and the neutral wire, and flow from positive and negative on the bridge rectifier, to the storage device 126.

In some embodiments, the controller 120 may include a display or other means of informing a user of the status of the device (e.g., an intelligent user interface that may incorporated into the prosthesis or provided via an application program on a smart phone). For example, an OHM meter may be used to indicate the current amount of voltage available in the storage device 126.

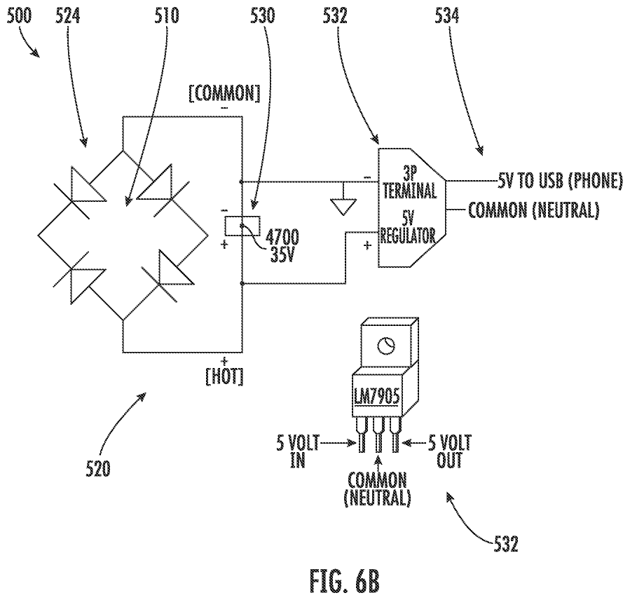

FIGS. 6A and 6B are simplified electrical schematics for a device 400, 500 in accordance with various embodiments of the disclosure. Referring to FIG. 6A, a basic set-up of the generator 410 and controller 420 are depicted. The enameled copper wire of the coils 418 on the generator 410 are electrically connected (either directly or indirectly) to the controller 420 (e.g., via soldering or electrical connectors as will be understood by those skilled in the art) to introduce electrical energy in the form of alternating current to the bridge rectifier 424. The bridge rectifier 424 comprises four (4) diodes (e.g., silicone, germanium, or pin) and converts the alternating current to a direct current, which is output to a capacitor 430 (e.g., a 5.5 volt, 0.47 F super capacitor) via the circuit board 422. The circuit board 422 is electrically connected to a storage device 428 (e.g., a rechargeable AA NiCad battery) that is in turn electrically connected to the prosthesis (e.g., a power supply or a motor thereof).

FIG. 6B depicts the electrical circuit in more detail. As shown, the device 500 includes a generator 510 electrically connected to the controller 520. The bridge rectifier 524 of the controller 520 is electrically connected to a capacitor 530 (e.g., 5 volts, 0.47 F) that is electrically connected to a voltage regulator 532 (e.g., 3-pin, 5 volt) to output 534 a fixed voltage to the battery and/or prosthesis. Components of the electric circuit may be sized dependent on the application and the energy anticipated to be generated and/or the energy requirements of the prostheses.

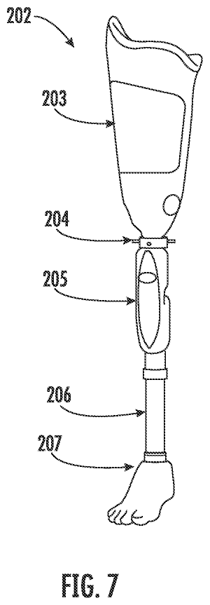

FIG. 7 depicts a typical leg prosthesis 202 for purposes of identifying the various components that the devices described herein may interact with for harvesting electrical energy. While a prosthetic leg is depicted, the devices disclosed herein may be used with essentially any prosthetic limb (e.g., transfemoral, transtibial, transradial, or transhumeral) or a portion thereof. As shown, the prosthesis includes a socket 203, which is the portion of the prosthesis that interfaces with a patient's limb stump or residual limb. The socket transmits forces from the prosthetic limb to the patient's body. Next is a rotator 204 and knee 205, which are present for a transfemoral prosthesis. In some embodiments, the rotator 204 and knee 205 do not interface with the device 100.

The main structural support for the prosthesis is called a pylon 206. The pylon 206 is the internal frame or skeleton of the prosthetic limb and is typically formed of metal rods or composites such as carbon fiber composites. The pylon 206 is typically enclosed by a cover made from a foam-like material that may be shaped and colored to match the recipient's skin tone to give the prosthetic limb a more lifelike appearance. Lastly, the prosthesis 202 includes a foot 207. The prosthesis 202 may include other components as needed, such as a suspension system, which may attach the prosthetic limb to the body. Depending on the type of limb, the devices (or portions thereof) described herein may be connected to the pylon 206 or the foot 207.

In order to further describe operation of an embodiment of the device 100, the normal human gait cycle, which will provide the electromotive force, is described herein. Generally, the human gait refers to the locomotion achieved through the movement of a person's limbs and is defined as bipedal, biphasic forward propulsion of the center of gravity of the human body, in which there are alternating sinuous movements of different segments of the body. With respect to a person's legs, in one instance the initial contact begins with foot fall (i.e., when the foot touches the ground) followed by a loading phase, where a person's weight is rapidly transferred onto the extended leg. This is the first period of double limb support. Next, the person's body is progressing over a single stable limb (i.e., mid-stance), followed by the body moving ahead of the leg on the ground, while the weight is being transferred to the forefoot (i.e., terminal stance). Swing limb advancement follows, where the leg is unloaded, the foot is up off of the ground, and the leg and foot are moved from behind the body to the front, taking the progressional step. The various swing phases are as follows: (a) pre-swing is the rapid de-weighting (unloading) of the leg on the ground, while the weight is shifting to the contralateral leg (i.e., the second period of double limb support); (b) initial swing is the start of advancement of the upper leg (thigh), where the foot is moving up and off of the floor; (c) mid swing involves the continued advancement of the upper leg as the knee starts to move into extension and the foot clears the ground; and (d) terminal swing where the knee comes to full extension and the leg prepares to make contact with ground.

With additional reference to the device 110 of FIGS. 2-6B, the harvesting of energy from the human gait cycle is detailed in accordance with the present disclosure. The movement of the limb causes the generator 110 to be rocked back and forth in a linear pattern resulting in the magnet 114 moving through the coils 118 to generate electrical energy in the form of alternating current. The electrical energy may be converted, transmitted, or stored by the controller 120. While the operation of an embodiment is described with respect to a user's legs, the devices described herein may also be incorporated into a prosthetic arm, for example, as will be understood by those skilled in the art. Because arm swing in human walking is a natural motion (i.e., typically each arm swings with the motion of the opposing leg), a generator 110 disposed within a prosthetic arm will also be rocked back and forth causing the magnet 114 to move through the coils 118 and generate electrical energy. This arrangement may be particularly advantageous during certain sports (e.g., race-walking or sprinting) where arm swing improves the stability and energy efficiency in human locomotion.

FIG. 8 depicts one exemplary installation of a device 300 within a prosthetic limb 302, in particular, the lower portion of a prosthetic leg. As shown in FIG. 8, the generator 310 portion of the device 300 is mounted to a portion of the prosthetic foot 352 (e.g., a carbon fiber plate) and encased by a foot cover 354. The generator 310 may be attached to the foot via an adhesive or mechanical fasteners 350 (e.g., screws, clamps, cable ties, hook and loop) either permanently or removably. The specific placement of the generator 310 may depend on the type of prosthesis. The generator 310 may be disposed on or in a portion of the limb that experiences regular movement. For example, the generator 310 may be disposed on or in a foot as it cycles through the human gait. In some embodiments, the device 300 may be retrofitted into an existing prosthesis, while in some embodiments, it may be incorporated into the prosthesis during manufacture thereof.

The controller 320 in FIG. 8 is shown attached to the pylon 306; however, the controller 320 may be positioned to best suit the prosthesis and/or the user. The controller 320 may be located adjacent the generator 310 such that wires running therebetween have a relatively clear path. Locating the controller 320 adjacent the generator 310 may reduce potential pinch points for the wires. The controller 320 may also be attached via an adhesive or mechanical fasteners 350 and, in some embodiments, may be disposed within a housing and then attached to the prosthesis 302.

In various embodiments, the device 300 may include additional components and functionalities. For example, in some embodiments, multiple generators 310 may be incorporated into the prosthesis 302. In some embodiments the device 300 may include 1, 2, 3, or more generators 310. In certain embodiments, a single generator 310 may include multiple motors. For example, a single generator 310 may include 1, 2, 3, or more motors. In embodiments, a piezo-electric generator 356 may replace or be used in addition to the magnetic linear generator 310. In some embodiments, the magnetic linear generator 310 generates electrical energy during the swing phase of the gait, while the piezo-electric generator generates electrical energy during foot fall (e.g., located in the heel such that it is impacted during the loading phase). In addition, the device 300 may include a microprocessor configured to control one or more functionalities of the device 300 or prosthesis 302, an indicator for displaying or transmitting information related to a status of the device 300 or prosthesis 302, or a mechanism that may be used to adjust the device 300 (e.g., voltage regulation).

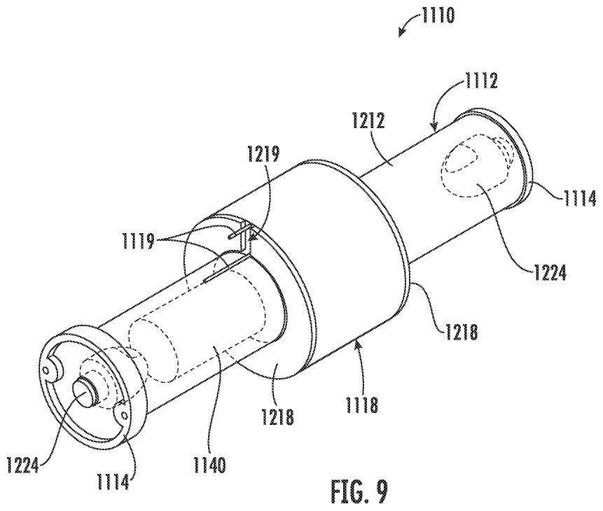

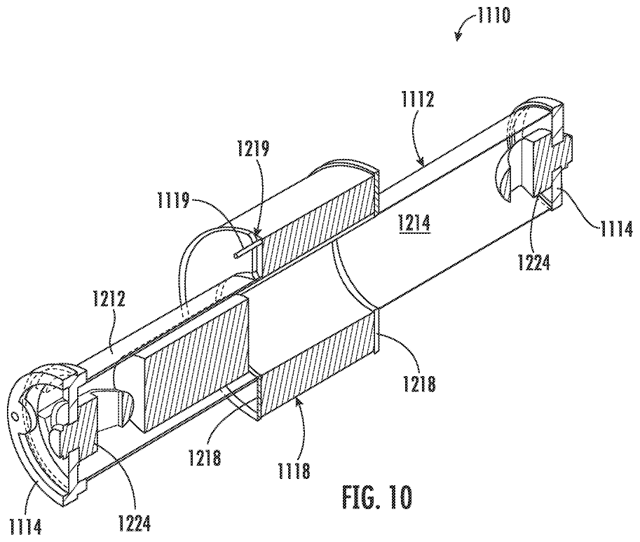

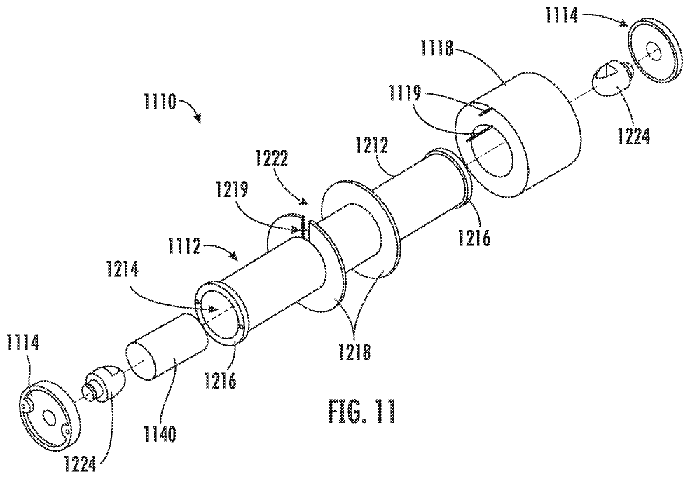

With reference to FIGS. 9-11, a magnetic linear motor 1110 provided in accordance with the present disclosure. The magnetic linear motor 1110 may be used in a similar manner to the motors detailed above and specifically, may be used in the devices and the methods detailed herein. The magnetic linear motor 1110 is configured to generate electrical energy in response to movement. For example, the magnetic linear motor 1110 may be associated with a prosthetic limb such that the magnetic linear motor 1110 generates electric energy in response to movement of a portion of the prosthetic limb and may generate electric energy in response to an electromotive force of the portion of the prosthetic limb.

The magnetic linear motor 1110 includes a body 1112, a coil 1118, and end caps 1114. The body 1112 may be a single monolithic or unitary element or may be formed of one or more parts that are joined together. The body 1112 includes a generally cylindrical tube 1212 that extends the length of the of the body 1112. The cylindrical tube 1212 is hollow such that a passage 1214 is defined therethrough.

The cylindrical tube 1212 may include a lip 1216 in each end portion thereof that is configured to secure a respective one of the endcaps 1114 thereto. The body 1112 may include one or more flanges 1218 that are disposed about the body 1112 adjacent a receiver 1222 on an outer surface of the body 1112. As shown, the body 1112 includes a pair of flanges 1218 that define a receiver 1222 therebetween about the outer surface of the body 1112. In some embodiments, the body 1112 includes multiple pairs of flanges 1218 with each pair defining a receiver 1222 therebetween. In certain embodiments, two pairs of flanges 1218 may also define a receiver 1222 therebetween such that two pairs of flanges 1218 (e.g., four flanges 1218) define a total of three receivers 1222. The body 1112 may include between 0 and 8 flanges 1218 and in some embodiments, the body 1112 may include more than eight flanges 1218. The lips 1216 and the flanges 1218 may be integrally, unitarily, or monolithically formed with the tube 1212. In particular embodiments, the lips 1216 or the flanges 1218 may be welded, adhered, or bonded to the tube 1212. In some embodiments, the lips 1216 or the flanges 1218 may be joined to the tube 1212 by one or more fasteners.

The coil 1118 is formed of wire that is wrapped around or about the body 1112. As shown, the magnetic linear motor 1110 includes a single coil 1118 wrapped around or about the body 1112 that is disposed in a receiver 1222 adjacent a flange 1218 or between a pair of flanges 1218. The wire forming the coil 1118 may be a single wire or may be multiple wires that are electrically coupled to one another. The wire forming the coil 1118 may be a copper wire and in certain embodiments, may be an enameled copper magnet wire. The wire may be a 22 or 32 gauge wire that is coated or uncoated. The coil 1118 may include leads 1119 that extend from the coil 1118 and electrically connect the coil 1118 to other components of a generator. In particular embodiments, one of the leads 1119 of the coil 1118 may extend from the coil 1118 to another coil 1118 and the other lead 1119 of the coil 1118 may extend to a controller of the generator, both of the leads 1119 of the coil 1118 may extend to a controller of the generator, or one leads 1119 of the coil 1118 may extend to another coil 1118 and the other lead 1119 of the coil 1118 may extend to still another coil 1118. It is contemplated that the magnetic linear motor 1110 may include between 1 and 4 coils 1118 and in some embodiments, may include more than 4 coils.

The one or more of the flanges 1218 may define a cutout 1219 that allow one or both of the leads 1119 to pass through the flange 1218. The cutout 1219 may allow for the lead 1119 to extend from the coil 1118 adjacent or along the outer surface of the body 1112.

The end caps 1114 are secured to the ends of the body 1112 such that the passage 1214 is closed. In some embodiments, the end caps 1114 seal the passage 1214. The end caps 1114 may interact with the lips 1216 to secure to the body 1112. Additionally or alternatively, the end caps 1114 may be adhered, bonded, or welded to the body 1112 at the lips 1216. In certain embodiments, the end caps 1114 may include a fastener to secure the end caps 1114 to the ends of the body 1112.

The magnetic linear motor 1110 further includes a magnet 1140 that is disposed within passage 1214 of the body 1112. The end caps 1114 are configured to secure the magnet 1140 within the passage 1214 while allowing the magnet 1140 to freely slide through the passage 1214 such that the magnet 1140 passes through the coil 1118. The magnet 1140 may be similar to the magnet 116b detailed above. As such, the magnet 1140 will not be detailed herein for brevity.

One or both of the end caps 1114 may include a bumper 1224 that is disposed within the passage 1214 and secured to the respective end cap 1114. The end cap 1114 may define an opening that receives a portion of the bumper 1224 therethrough to secure the bumper 1224 to the end cap 1114. The bumpers 1224 are configured to absorb mechanical energy as the magnet 1140 engages the bumper 1224 and urge the magnet 1140 away from the end cap 1114 to which the bumper 1224 is secured and towards the other bumper 1224 such that the magnet 1140 passes through the coil 1118. The bumpers 1224 may include a spring portion that elastically deforms and stores mechanical energy as it deforms and releases the storage mechanical energy to urge the magnet 1140 towards the opposite bumper 1224 as the bumper 1224 returns to its original shape. The bumpers 1224 may function in a manner similar to the second and third magnets detailed above.

In a manner similar to the magnetic linear motor 110, as the magnet 1140 passes through the coil 1118, the magnet 1140 induces electrical energy in the coil 1118 that passes to a controller in the form of alternating current. The body 1112 may be attached to or disposed within a prosthetic limb such that the magnet 1140 slides within the passage 1214 of the body 1112 in response to movement of the prosthetic limb. For example, the magnet may slide within the passage 1214 as the prosthetic limb is in a “swing” portion of a human gait as detailed above.

As noted above, it is within the scope of this disclosure that the magnetic linear motor 1110 may be used in conjunction with or as a replacement for the magnetic linear motor 110 detailed above. Specifically, the magnetic linear motor 1110 may be used with the device 100, 300, the controller 120, the prosthesis 202, and/or the methods detailed herein.

Having now described some illustrative embodiments of the disclosure, it should be apparent to those skilled in the art that the foregoing is merely illustrative and not limiting, having been presented by way of example only. Numerous modifications and other embodiments are within the scope of one of ordinary skill in the art and are contemplated as falling within the scope of the disclosure. In particular, although many of the examples presented herein involve specific combinations of method acts or system elements, it should be understood that those acts and those elements may be combined in other ways to accomplish the same objectives.

Furthermore, those skilled in the art should appreciate that the parameters and configurations described herein are exemplary and that actual parameters and/or configurations will depend on the specific application in which the systems and techniques of the disclosure are used. Those skilled in the art should also recognize or be able to ascertain, using no more than routine experimentation, equivalents to the specific embodiments of the disclosure. It is, therefore, to be understood that the embodiments described herein are presented by way of example only and that, within the scope of any appended claims and equivalents thereto; the disclosure may be practiced other than as specifically described.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to any claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish claim elements.

What is claimed is:

1. A device to harvest electrical energy from movement of a prosthetic limb, the device comprising:
a generator adapted to attach to a prosthetic limb, the generator comprising a first motor configured to generate electrical energy in response to movement of a portion of the prosthetic limb; and
a controller electrically connected to the generator, the controller configured to provide electrical energy to a power supply of the prosthetic limb.

2. The device according to claim 1, wherein the controller is configured to charge the power supply of the prosthetic limb.

3. The device according to claim 1, wherein the first motor is configured to be disposed within the portion of the prosthetic limb.

4. The device according to claim 1, wherein the first motor comprises:
a hollow tubular body;
at least one coil of wire wrapped about the body; and
a first magnet slidably disposed within the body, the first magnet configured to slide within the body and through the at least one coil of wire in response to movement of the portion of the prosthetic limb such that electrical energy is generated in the at least one coil of wire.

5. The device according to claim 4, wherein the first magnet is configured to slide within the body during a swing phase of a human gait.

6. The device according to claim 4, wherein the first motor comprises:
a first end cap closing a first end of the body;
a second end cap closing a second end of the body opposite the first end;
a second magnet disposed in the first end cap; and
a third magnet disposed in the second end cap, the second magnet and the third magnet oriented such that polarities thereof oppose a polarity of the first magnet slidably disposed therebetween.

7. The device according to claim 6, wherein the first magnet, the second magnet, or the third magnet comprise a neodymium magnet.

8. The device according to claim 4, wherein the first motor comprises:
a first end cap closing a first end of the body, the first end cap including a first bumper, the first bumper secured to the first end cap and disposed within the body; and
a second end cap closing a second end of the body opposite the first end, the second end cap including a second bumper, the second bumper secured to the second end cap and disposed within the body, the first bumper and the second bumper configured to engage the first magnet.

9. The device according to claim 4, wherein the at least one coil of wire forms four separate coils of wire wrapped about the body, the four separate coils being in electrical communication with one another and spaced equidistant along the body.

10. The device according to claim 3, wherein the first motor is configured to generate electrical energy in the form of alternating current.

11. The device according to claim 10, wherein the controller is configured to convert the alternating current from the generator to direct current and to provide the direct current to the power supply of the prosthetic.

12. The device according to claim 11, wherein the controller comprises an electrical storage medium configured to receive and store electrical energy from the first motor, the electrical storage medium configured to output stored electrical energy to the power supply of the prosthetic limb.

13-16. (canceled)

17. A prosthetic limb comprising:
a first portion;
a power supply; and
a generator comprising:
a first motor configured to generate electrical energy in response to movement of the first portion; and
a controller electrically connected to the generator, the controller configured to provide electrical energy to the power supply.

18. The prosthetic limb according to claim 17, wherein the first motor is a magnetic linear motor or a piezo-electric motor.

19. The prosthetic limb according to claim 17, wherein the first motor is disposed within the first portion.

20. The prosthetic limb according to claim 17, wherein the generator comprises a second motor configured to generate electrical energy in response to movement of the prosthetic limb.

21. The prosthetic limb according to claim 20, wherein the first motor and the second motor are disposed in the first portion.

22. The prosthetic limb according to claim 20, further comprising a second portion, the second motor configured to generate electrical energy in response to movement of the second portion of the prosthetic limb.

23. The prosthetic limb according to claim 22, further comprising a foot, the second portion being a heel of the foot.

24. The prosthetic limb according to claim 22, further comprising a leg segment, the first portion being a section of the leg segment.

25-29. (canceled)
